(19) United States
(12) Patent Application Publication
(10) Pub. No.: US 2025/0391499 A1
(43) Pub. Date: Dec. 25, 2025

(54) CANCER STAGING METHOD AND ELECTRONIC DEVICE

(71) Applicants: Beijing BOE Technology Development Co., Ltd., Beijing (CN); BOE Technology Group Co., Ltd., Beijing (CN)

(72) Inventors: Yang SONG, Beijing (CN); Yaqi ZHANG, Beijing (CN)

(21) Appl. No.: 18/996,906

(22) PCT Filed: May 24, 2024
(86) PCT No.: PCT/CN2024/095217

§ 371 Date: Jan. 17, 2025

(30) Foreign Application Priority Data
Jun. 29, 2023 (CN) 202310790387.4

(51) Int. Cl. G16B 20/00; G16B 40/20

(52) U.S. Cl. CPC G16B 20/00; G16B 40/20

(57) ABSTRACT

Provided are a cancer staging method and an electronic device. The method includes: acquiring a methylation data set of a target subject; and inputting the methylation data set into a staging prediction model, and outputting a cancer staging value of the target subject, the staging prediction model being obtained by means of integration of n classification models, model parameters of the n classification models being obtained by means of performing optimization by using a grid retrieval method, the n classification models being obtained from m classification models by means of performing screening by using a cross validation method, and the m classification models being obtained by means of performing independent training by using the same data set, wherein m>n, m is an integer greater than 2, and n is an integer greater than 1.

### CROSS REFERENCE TO RELATED APPLICATION

This application is a national phase entry under 35 U.S.C. § 371 of International Application No. PCT/CN2024/095217, filed on May 24, 2024, which claims priority to Chinese Patent Application No. 202310790387.4, filed with the China National Intellectual Property Administration on Jun. 29, 2023, and entitled “CANCER STAGING METHOD AND ELECTRONIC DEVICE”, both of which are hereby incorporated by reference in their entireties.

### TECHNICAL FIELD

The present disclosure relates to the field of biological information technology, particularly to a cancer staging method and electronic device.

### BACKGROUND

Cancer staging is a method that determines the degree of cancer development and spread. Accurately staging cancer is beneficial for developing the most reasonable treatment plan for cancer patients and effectively assessing the prognosis of cancer.

In the related art, clinical and pathological examinations are usually used for cancer staging, but only clinical examinations (blood tests, radiographic examinations, endoscopic examinations, etc.) provide limited information for cancer staging judgment and have limitations. Although pathological examinations can more accurately determine staging, surgical intervention is required to obtain pathological sections, and not all tumors require surgical treatment. Additionally, some patients may have undergone chemotherapy and radiation therapy before pathological examination, which may underestimate the true staging of the tumor to some extent. Therefore, the use of pathological examination methods still has limitations. Moreover, different cancers have different staging systems, and some cancers do not have suitable staging methods. Conventional staging methods are not universal.

### SUMMARY

The present disclosure provides a cancer staging method and electronic device for predicting cancer staging corresponding to an input methylation dataset using a staging prediction model integrated from multiple classification models, ensuring prediction accuracy while improving the universality of cancer staging.

In a first aspect, embodiments of the present disclosure provide a cancer staging method, including:
obtaining a methylation dataset of a target object;
inputting the methylation dataset into a staging prediction model to output a cancer staging value of the target object;
wherein the staging prediction model is obtained by integrating n classification models, model parameters of the n classification models are obtained by optimization using a grid search method, the n classification models are obtained by filtering from m classification models using a cross-validation method, the m classification models are obtained by independent training using a same dataset, wherein m>n, m is an integer greater than 2, and n is an integer greater than 1.

As an optional embodiment, the obtaining the methylation dataset of the target object includes:
obtaining methylation data corresponding to a target methylation site of the target object, and determining the methylation dataset based on the methylation data corresponding to the target methylation site;
wherein the target methylation site is determined based on a degree of difference in methylation data of a same methylation site corresponding to different cancer staging values, or a correlation between methylation data corresponding to a methylation site and different cancer staging values.

As an optional embodiment, the staging prediction model further includes a normalization model, and the normalization model is used to normalize methylation data in the methylation dataset by means of de-meaning and variance normalization.

As an optional embodiment, the staging prediction model further includes a dimensionality reduction model, and the dimensionality reduction model is used to perform dimensionality reduction on methylation data in the methylation dataset according to a principal component analysis (PCA) method.

As an optional embodiment, the staging prediction model is determined by:
independently training the m classification models separately using the dataset, and determining classification accuracies of the m classification models;
filtering out k classification models from the m classification models according to the classification accuracies.

As an optional embodiment, after filtering out the k classification models from the m classification models according to the classification accuracies, the method further includes:
filtering out the n classification models from the k classification models using the cross-validation method, wherein m≥k≥n; and/or
adjusting the model parameters of the filtered classification models using the grid search method.

As an optional embodiment, the filtering out the n classification models from the k classification models using the cross-validation method includes:
cross-validating the k classification models using the dataset to obtain evaluation index values of the k classification models;
filtering out the n classification models from the k classification models based on the evaluation index values of the k classification models.

As an optional embodiment, after obtaining the dataset, the method further includes:
filtering out methylation data corresponding to a target methylation site from methylation data contained in the dataset, and independently training the m classification models using the filtered methylation data.

As an optional embodiment, the dataset includes sample sets of different target objects, the sample sets each includes methylation data corresponding to each methylation site, and one of the sample sets corresponds to one of the cancer staging values;
wherein the target methylation site is determined by:
dividing the dataset into a plurality of subsets according to the cancer staging values corresponding to the sample sets, wherein one of the subsets corresponds to one of the cancer staging values, and sample sets contained in different subsets correspond to different cancer staging values;
for each methylation site, determining the target methylation site according to the methylation data in the plurality of subsets corresponding to the methylation site, respectively.

As an optional embodiment, the determining the target methylation site according to the methylation data in the plurality of subsets corresponding to the methylation site, respectively, includes:
when the dataset is divided into two subsets, determining, for each methylation site, the target methylation site according to a degree of difference in the methylation data corresponding to the methylation site in each of the two subsets; or.
when the dataset is divided into more than two subsets, determining, for each methylation site, the target methylation site according to a correlation between the methylation data corresponding to the methylation site in each subset and the cancer staging value corresponding to the each subset.

As an optional embodiment,
the degree of difference in the methylation data corresponding to the methylation site in each of the two subsets is determined by:
performing a rank sum test on the methylation data corresponding to the methylation site in each of the two subsets, and determining the degree of difference according to a rank sum check value; or
the correlation between the methylation data corresponding to the methylation site in each subset and the cancer staging value corresponding to the each subset is determined by:
performing a chi-square test on the methylation data corresponding to the methylation site in each subset and the cancer staging value corresponding to the subset, and determining the correlation according to a value of the chi-square test.

As an optional embodiment, the plurality of subsets include more than two subsets; the dividing the dataset into the plurality of subsets according to the cancer staging values corresponding to the sample sets, the method further includes:
when a subset in which a quantity of sample sets is lower than a threshold value exists in the divided plurality of subsets, merging the subset into another subset according to the cancer staging value corresponding to the subset, determining a cancer staging value corresponding to the another subset after the merging, and determining the target methylation site and training of the classification model using the subset after the merging; wherein the cancer staging value corresponding to the another subset is closest to the cancer staging value corresponding to the subset.

As an optional embodiment, the method further includes:
removing, with respect to a sample set of the target object, methylation data corresponding to a methylation site of normal tissue adjacent to a cancer in the sample set, and/or, methylation data corresponding to a methylation site containing a missing value.

As an optional embodiment, the staging prediction model is obtained by integrating according to the following:
integrating the n classification models into the staging prediction model by using outputs of n−1 classification models as an input of an nth classification model;
wherein inputs of the n−1 classification models are the dataset, and an output of the nth classification model is a cancer staging value corresponding to the dataset.

In a second aspect, embodiments of the present disclosure further provide an electronic device, including a processor and a memory, wherein the memory is configured for storing programs executable by the processor, and the processor is configured for reading the programs in the memory to perform:
obtaining a methylation dataset of a target object;
inputting the methylation dataset into a staging prediction model to output a cancer staging value of the target object;
wherein the staging prediction model is obtained by integrating n classification models, model parameters of the n classification models are obtained by optimization using a grid search method, the n classification models are obtained by filtering from m classification models using a cross-validation method, the m classification models are obtained by independent training using a same dataset, wherein m>n, m is an integer greater than 2, and n is an integer greater than 1.

As an optional embodiment, the processor is specifically configured for:
obtaining methylation data corresponding to a target methylation site of the target object, and determining the methylation dataset based on the methylation data corresponding to the target methylation site;
wherein the target methylation site is determined based on a degree of difference in methylation data of a same methylation site corresponding to different cancer staging values, or a correlation between methylation data corresponding to a methylation site and different cancer staging values.

As an optional embodiment, the staging prediction model further includes a normalization model, and the normalization model is used to normalize methylation data in the methylation dataset by means of de-meaning and variance normalization.

As an optional embodiment, the staging prediction model further includes a dimensionality reduction model, and the dimensionality reduction model is used to perform dimensionality reduction on methylation data in the methylation dataset according to a principal component analysis (PCA) method.

As an optional embodiment, the processor is specifically configured for determining the staging prediction model by:
independently training the m classification models separately using the dataset, and determining classification accuracies of the m classification models;
filtering out k classification models from the m classification models according to the classification accuracies.

As an optional embodiment, after filtering out the k classification models from the m classification models according to the classification accuracies, the processor is specifically configured for:
filtering out the n classification models from the k classification models using the cross-validation method, wherein m≥k≥n; and/or
adjusting the model parameters of the filtered classification models using the grid search method.

As an optional embodiment, the processor is specifically configured for:
cross-validating the k classification models using the dataset to obtain evaluation index values of the k classification models;
filtering out the n classification models from the k classification models based on the evaluation index values of the k classification models.

As an optional embodiment, after obtaining the dataset, the processor is specifically configured for:
filtering out methylation data corresponding to a target methylation site from methylation data contained in the dataset, and independently training the m classification models using the filtered methylation data.

As an optional embodiment, the dataset includes sample sets of different target objects, the sample sets each includes methylation data corresponding to each methylation site, and one of the sample sets corresponds to one of the cancer staging values;
wherein the processor is specifically configured for determining the target methylation site by:
   dividing the dataset into a plurality of subsets according to the cancer staging values corresponding to the sample sets, wherein one of the subsets corresponds to one of the cancer staging values, and sample sets contained in different subsets correspond to different cancer staging values;
   for each methylation site, determining the target methylation site according to the methylation data in the plurality of subsets corresponding to the methylation site, respectively.

As an optional embodiment, the processor is specifically configured for:
when the dataset is divided into two subsets, determining, for each methylation site, the target methylation site according to a degree of difference in the methylation data corresponding to the methylation site in each of the two subsets; or
when the dataset is divided into more than two subsets, determining, for each methylation site, the target methylation site according to a correlation between the methylation data corresponding to the methylation site in each subset and the cancer staging value corresponding to the each subset.

As an optional embodiment,
wherein the processor is specifically configured for determining the degree of difference in the methylation data corresponding to the methylation site in each of the two subsets by:
performing a rank sum test on the methylation data corresponding to the methylation site in each of the two subsets, and determining the degree of difference according to a rank sum check value; or
the correlation between the methylation data corresponding to the methylation site in each subset and the cancer staging value corresponding to the each subset is determined by:
performing a chi-square test on the methylation data corresponding to the methylation site in each subset and the cancer staging value corresponding to the subset, and determining the correlation according to a value of the chi-square test.

As an optional embodiment, the plurality of subsets include more than two subsets; the dividing the dataset into the plurality of subsets according to the cancer staging values corresponding to the sample sets, the processor is specifically configured for:
when a subset in which a quantity of sample sets is lower than a threshold value exists in the divided plurality of subsets, merging the subset into another subset according to the cancer staging value corresponding to the subset, determining a cancer staging value corresponding to the another subset after the merging, and determining the target methylation site and training of the classification model using the subset after the merging; wherein the cancer staging value corresponding to the another subset is closest to the cancer staging value corresponding to the subset.

As an optional embodiment, the processor is specifically configured for:
removing, with respect to a sample set of the target object, methylation data corresponding to a methylation site of normal tissue adjacent to a cancer in the sample set, and/or, methylation data corresponding to a methylation site containing a missing value.

As an optional embodiment, the staging prediction model is obtained by integrating according to the following:
integrating the n classification models into the staging prediction model by using outputs of n−1 classification models as an input of an nth classification model;
wherein inputs of the n−1 classification models are the dataset, and an output of the nth classification model is a cancer staging value corresponding to the dataset.

In a third aspect, embodiments of the present disclosure further provide a computer storage medium, storing computer programs, wherein the programs when executed by a processor implement steps of the method in the first aspect.

The aspects or other aspects disclosed herein will be more concise and understandable in the description of the following embodiments.

### BRIEF DESCRIPTION OF FIGURES

In order to more clearly illustrate the technical solutions in the embodiments of the present disclosure, the accompanying drawings to be used in the description of the embodiments will be briefly introduced below, and it will be obvious that the accompanying drawings in the following description are only some of the embodiments of the present disclosure, and other accompanying drawings can be obtained based on these drawings for the person of ordinary skill in the field, without giving creative labor power.

FIG. 1 shows a flowchart of a specific implementation of a cancer staging method provided by embodiments of the present disclosure.

FIG. 2 shows a schematic diagram of a data form after preprocessing of a dataset provided by embodiments of the present disclosure.

FIG. 3 shows a schematic diagram of a p-value for differential analysis of methylation data corresponding to each methylation site provided by embodiments of the present disclosure.

FIG. 4 shows a distribution diagram of methylation data before unstandardized processing provided by embodiments of the present disclosure.

FIG. 5 shows a distribution diagram of methylation data after normalization and dimensionality reduction provided by embodiments of the present disclosure.

FIGS. 6A to 6E are schematic diagrams of a cross-validated classification model evaluation provided by embodiments of the present disclosure.

FIG. 7 shows a flowchart of a multi-model integration provided by embodiments of the present disclosure.

FIG. 8 shows a graph of ROC_AUC indicator evaluation curve of a staging prediction model provided by embodiments of the present disclosure.

FIG. 9 shows a graph of a result of cancer staging prediction of a liver cancer patient using a staging prediction model provided by embodiments of the present disclosure.

FIG. 10 shows a flow chart of a specific implementation of a cancer staging method provided by embodiments of the present disclosure.

FIG. 11 shows a schematic diagram of a cancer staging device provided by embodiments of the present disclosure.

FIG. 12 shows a schematic diagram of an electronic device provided by embodiments of the present disclosure.

### DETAILED DESCRIPTION

In order to make the objects, technical solutions and advantages of the present disclosure clearer, the present disclosure will be described in further detail in the following in connection with the accompanying drawings, and it is obvious that the described embodiments are only a part of the embodiments of the present disclosure, and not all of the embodiments. Based on the embodiments in the present disclosure, all other embodiments obtained by a person of ordinary skill in the art without making creative labor are within the scope of protection of the present disclosure.

The term “and/or” in the embodiments of the present disclosure, describing an association relationship of the associated objects, indicates that three kinds of relationships can exist, for example, A and/or B, which can be expressed as: the existence of A alone, the existence of both A and B, and the existence of B alone. The character “/” generally indicates that the associated objects are in an “or” relationship.

In embodiments of the present disclosure, the term “methylation”, in a biological system, methylation is catalyzed by an enzyme, methylation involves heavy metal modification, regulation of gene expression, regulation of protein function, and ribonucleic acid (RNA) processing, and gene methylation is the most prominent form of epigenetic inheritance, and methylation is capable of influencing the development of tumorigenesis.

In embodiments of the present disclosure, the term “cancer staging” is a method for determining the degree of development and spread of cancer, and the staging of cancer affects the treatment plan of the patient and the judgment of the prognosis of the cancer.

In embodiments of the present disclosure, the term “Principal Component Analysis (PCA)” is commonly used for dimensionality reduction of high-dimensional data, and can be used to extract main features of the data.

In embodiments of the present disclosure, the term “Grid Search” is a model hyper-parameter optimization technique, which is commonly used to optimize three or fewer hyper-parameters, and is essentially an exhaustive method.

In embodiments of the present disclosure, the term “model integration” refers to combining multiple weakly-supervised models to obtain a better and more comprehensive strongly-supervised model, with the underlying concept of integrating the models being that, even if one weak classifier obtains an incorrect prediction, other weak classifiers can correct the error.

In embodiments of the present disclosure, the application scenarios are for the purpose of more clearly illustrating the technical solutions of the embodiments of the present disclosure and do not constitute a limitation of the technical solutions provided by the embodiments of the present disclosure, and a person of ordinary skill in the art may know that, with the emergence of new application scenarios, the technical solutions provided by the embodiments of the present disclosure are equally applicable to similar technical problems. Here, in the description of the present disclosure, unless otherwise specified, “plurality” means two or more.

Cancer staging is a method for determining the degree of development and spread of a cancer, and accurately staging the cancer is helpful for formulating the most reasonable treatment plan for a cancer patient and effectively determining the prognosis of the cancer. Among hematological tumors, Ann Arbor staging system is used for lymphoma and Hodgkin's lymphoma; among solid tumors, TNM (Tumor Node Metastasis) staging is the most common staging system, but there are many differences depending on tumor types, and commonly applied tumor types include: breast cancer, colon cancer, kidney cancer, laryngeal cancer, hepatocellular carcinoma, lung cancer, prostate cancer, skin cancer, bladder cancer and so on. According to the degree of harm of the tumor, it can be divided into stages I, II, III and IV. Since the related art mostly utilizes clinical and pathological examinations and other methods for cancer staging, the drawback is that when only clinical examination (blood test, radiological examination, endoscopic examination, etc.) is carried out, the implementation method is indirect observation, and the information for judging the cancer staging is limited, and has limitations. Although pathological examination can determine the stage more accurately, it requires surgical operation to obtain pathological sections, not all tumors require surgical treatment, and some patients may have undergone chemotherapy and radiotherapy before pathological examination, which will underestimate the true staging of the tumor to a certain extent, so the method of using pathological examination still has limitations. Secondly, different cancers have different staging systems, and some cancers do not have appropriate staging methods; staging is highly subjective and conventional staging methods are not universal. In addition to clinical systems, there are also methods for cancer staging based on methylation data, but these methods usually consider only a few methylation sites and have a single classification model that fails to effectively utilize most of the methylation data in the genome.

The embodiments of the present disclosure provide a cancer staging method for predicting a cancer stage corresponding to an input methylation dataset by a staging prediction model obtained by integrating a plurality of classification models, the staging prediction model in the embodiments of the present disclosure is independently trained by m classification models, and filters out n classification models using cross-validation, and model parameters of these n classification models are obtained by optimizing using a grid search method, which can effectively capture the complex relationship between methylation data and cancer staging, and improve the accuracy of cancer staging prediction; the use of methylation site information for cancer staging prediction is applicable to the vast majority of cancer types as compared to current clinical staging methods, and can effectively differentiate between the cancer staging types of different patients, and improve the universality of cancer staging prediction.

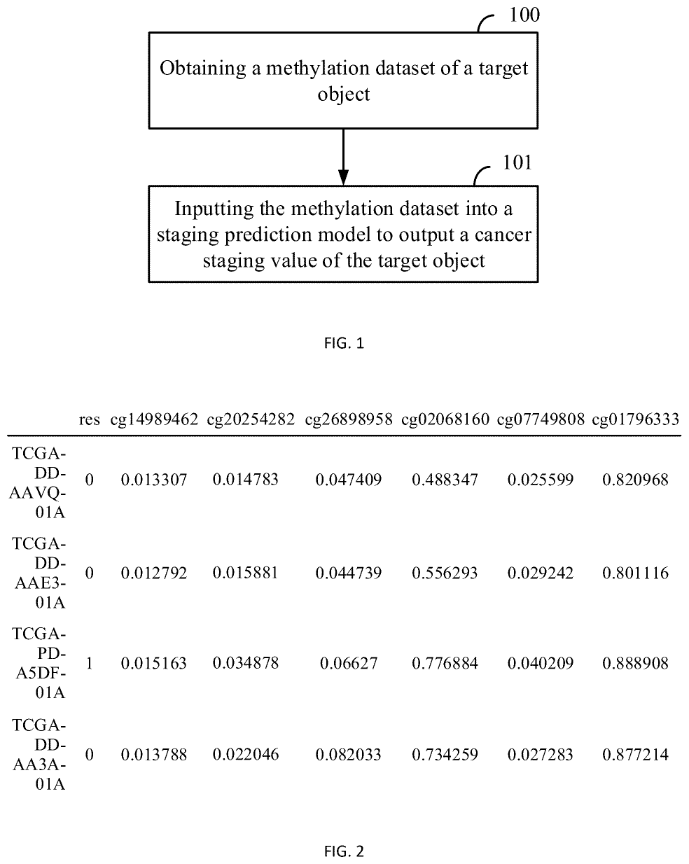

As shown in FIG. 1, a specific implementation process of a cancer staging method provided by the embodiments of the present disclosure is shown below.

Step 100: obtaining a methylation dataset of a target object.

The target object in the embodiments of the present disclosure includes, but is not limited to, human tumor tissue.

In the implementations, the methylation dataset in the embodiments of the present disclosure includes methylation data corresponding to a plurality of methylation sites, where one methylation site of a sample corresponds to one methylation data. The methylation data in the embodiments of the present disclosure includes a methylation level value.

In some embodiments, the methylation dataset of the target object is determined by:
obtaining methylation data corresponding to a target methylation site of the target object, and determining the methylation dataset based on the methylation data corresponding to the target methylation site.

Here, the target methylation site is determined based on a degree of difference in methylation data of the same methylation site corresponding to different cancer staging values, or based on a correlation between methylation data corresponding to the methylation site and different cancer staging values.

In the implementations, the methylation dataset of the target object in the embodiments of the present disclosure includes the methylation data corresponding to the target methylation site, and the target methylation site is filtered out of various methylation sites, and since the methylation data corresponding to the target methylation site more accurately represents the features of the cancer staging, the methylation dataset formed by using the methylation data corresponding to the target methylation site can more accurately make a prediction of the cancer staging and improve the accuracy of prediction.

It should be noted that the number of target methylation sites is less than or equal to the number of individual methylation sites contained in the target object itself.

Step 101, inputting the methylation dataset into a staging prediction model to output a cancer staging value of the target object.

Here, the staging prediction model is obtained based on the integration of n classification models, the model parameters of the n classification models are obtained by optimization using the grid search method, the n classification models are obtained by filtering from m classification models using the cross-validation method, the m classification models are obtained by independent training using the same dataset, where m>n, m is an integer greater than 2, and n is an integer greater than 1.

In the implementations, the staging prediction model in the embodiments of the present disclosure is used to perform cancer staging prediction on the input methylation dataset, on the one hand, since the staging prediction model of the embodiments of the present disclosure is integrated by a plurality of classification models, and model integration refers to the combination of a plurality of weakly-supervised models with a view to obtain a better and more comprehensive strongly-supervised model, and the concept underlying the model integration is that even if a certain weak classifier obtains an incorrect prediction, other weak classifiers can correct the error. Thus the accuracy of the predictions of the staging prediction model is improved by model integration. On the other hand, the staging prediction model in the embodiments of the present disclosure contains n classification models that are filtered from m classification models using cross-validation, and cross-validation can assess whether the m classification models perform well on the entire dataset, which is used to assess the stability and generalization ability of the classification models, for example, when the AUC metrics of one classification model among the m classification models for all folds of the cross-validation fluctuates greatly, then that classification model is excluded, i.e., it is not selected as one of the n classification models. On the last aspect, the model parameters of the n classification models ultimately selected in the embodiments of the present disclosure are obtained by optimization using grid search, where the grid search is a model hyper-parameter optimization technique commonly used to optimize three or a smaller number of hyper-parameters, and is essentially an exhaustive method. The model parameters of the n classification models are adjusted using grid search to select the model parameters that obtain the best performance of the classification models based on the n classification models that have been selected. The embodiments of the present disclosure make the staging prediction model optimal in terms of accuracy, stability, and predictive ability for new datasets by means of collaborative execution of independent training of multiple classification models, cross-validation, and grid search tuning.

In some embodiments, the staging prediction model further includes a normalization model, and the normalization model is used to normalize the methylation data in the methylation dataset by means of de-mean and variance normalization.

In the implementations, since the distribution of the methylation data of the plurality of target methylation sites belongs to a skewed distribution, in order to make the methylation rate of the methylation data of the plurality of target methylation sites uniform in scale, the methylation data of each target methylation site can be standardized, so as to make the methylation data of the processed target methylation site each as close as possible to a normal distribution. Thereby, the subsequent prediction based on the methylation data of each target methylation site can be more accurate.

In some embodiments, the staging prediction model further includes a dimensionality reduction model, and the dimensionality reduction model is used to perform dimensionality reduction on the methylation data in the methylation dataset according to the PCA method.

In the implementations, PCA transforms the original data into a set of linearly independent representations in each dimension by linear transformation, which can be used to extract the main feature components of the data, and is commonly used for dimensionality reduction of high-dimensional data. The PCA extracts the main features in the methylation dataset formed by each target methylation data, thereby obtaining a methylation dataset containing the main features, so that when subsequent prediction is performed based on the methylation dataset containing the main features, the amount of data to be calculated is reduced, and the prediction speed and efficiency of the algorithm are improved.

Optionally, the staging prediction model in the embodiments of the present disclosure includes, but is not limited to, performing standardization processing and dimensionality reduction processing on the methylation dataset, and the methylation dataset after the standardization processing and dimensionality reduction processing is used to perform the prediction. The embodiments of the present disclosures do not impose excessive limitations on the sequence of the standardization processing and the dimensionality reduction processing.

In some embodiments, the embodiments of the present disclosure determine the staging prediction model in the following manner.

Step 1, using the dataset to independently train the m classification models respectively to determine a classification accuracy of each of the m classification models.

In the implementations, the dataset includes a sample set(s) of different target objects, the sample set includes methylation data corresponding to each methylation site; a sample set corresponds to a cancer staging value; a target object corresponds to a sample set, a sample set includes methylation data corresponding to a plurality of methylation sites.

In the implementations, in the process of training the classification model, a training set in the dataset is utilized for training, and the training set includes the sample set.

Optionally, after obtaining the dataset and before training the classification model using the dataset, the methylation data in the dataset can also be preprocessed, and the preprocessing method includes, but is not limited to, a combination of any one or more of the following ways:
Way 1a, after obtaining the sample set, the methylation data corresponding to the methylation site of normal tissue adjacent to the cancer in the sample set can also can be removed for the sample set of the target object;
Way 1b, after obtaining the sample set, the methylation data corresponding to the methylation site in the sample set including a missing value (NA) can be removed for the sample set of the target object.

In embodiments, the sample sets can be obtained from a public database TCGA, each sample set corresponds to a cancer staging value, and the methylation data corresponding to the methylation site of normal tissue adjacent to the cancer and the methylation data corresponding to the methylation site of tumor tissue including a missing value can be removed.

In some embodiments, after obtaining the dataset, the embodiments of the present disclosure can also filter the training set in the dataset again to filter the methylation data corresponding to the target methylation site representing the features of the cancer staging to improve the prediction accuracy in the following manner.
Way 1c, filtering out the methylation data corresponding to the target methylation site from the methylation data contained in the training set in the dataset, and independent training the m classification models using the filtered methylation data. Here, the filtered methylation data is the methylation data corresponding to the filtered target methylation site.

In embodiments, after obtaining the sample set and removing the methylation data corresponding to the methylation site of normal tissue adjacent to the cancer and the methylation data corresponding to the methylation site including the missing value, the methylation data corresponding to the target methylation site is then filtered out of the sample set after the removal, so as to form a dataset using the filtered sample set.

In some embodiments, the target methylation site is determined based on the training set in the dataset, and the target methylation site can be determined by the following process.

Process c1), dividing the dataset into a plurality of subsets according to the cancer staging values corresponding to the sample set, where a subset corresponds to a cancer staging value, and sample sets contained in different subsets correspond to different cancer staging values.

In the implementations, the dataset can be divided into two subsets or more than two subsets. For example, one subset corresponds to one cancer staging value, and the sample set of stage I is divided into one subset, the sample set of stage II is divided into one subset, the sample set of stage III is divided into one subset, and the sample set of stage IV is divided into one subset, thereby dividing the dataset into four subsets eventually. It is also possible to divide the sample sets of stage I and stage II into one subset, and the sample sets of stage III and stage IV into one subset, i.e., to divide the dataset into two subsets, to set the cancer staging value corresponding to one subset of the sample sets including stage I and stage II into a pre-stage, and to set the cancer staging value corresponding to one subset of the sample sets including stage III and stage IV into a post-stage. The embodiments of the present disclosure do not impose excessive limitations on the division of the dataset, and the details can be adjusted according to the actual situation.

Optionally, when the dataset is divided into a plurality of subsets according to the cancer staging values corresponding to the sample sets, if the divided plurality of subsets includes more than two subsets, merging among the subsets can also be performed, as shown in the following steps:
if in the divided plurality of subsets, there exists a subset in which the number of sample sets is lower than a threshold value, then according to the cancer staging value corresponding to the subset, merging the subset into another subset and determining the cancer staging value corresponding to the another subset after merging, to determine the target methylation site and the training of the classification model using each of the merged subsets; where the cancer staging value of the another subset is closest to the cancer staging value corresponding to the subset.

In the implementations, when there are more than two subsets, if there is a size imbalance in the data volume of the methylation data between the different subsets, the different subsets can be merged. For example, the dataset includes a sample set of different hepatocellular carcinoma patients and corresponding cancer staging values, where the number of stage I cases (the number of sample sets) is 212, the number of stage II cases is 107, and the total number of stage III and IV cases is 150, removing some of the methylation sites with missing methylation data and removing the methylation data of the normal samples, and grouping the sample sets according to the cancer staging values, i.e., the dataset is split. Since the number of cases of stage Ill and IV are scattered, and the number of cases in some stages is small, there is an imbalance in the sample set, so the cases of stage III and IV are combined into late cancer, while the number of cases of stage I and II are combined into early cancer, so that the early cancer contains a total of 265 cases, and the late cancer contains a total of 115 cases.

Optionally, after dividing the dataset into a plurality of subsets, the dataset can also be formatted so as to facilitate the execution of the next process, for example, the dataset can be transposed so that the first columns are all cancer stage values, and the second columns and onwards are methylation data corresponding to methylation sites, as shown in FIG. 2, which shows a data form after preprocessing the dataset provided by the embodiments of the present disclosure.

After preprocessing the dataset, the preprocessed dataset is split into a training set and a testing set in an 8:2 ratio. In a specific implementation, the dataset is first divided in stages, i.e., after the dataset is divided into a plurality of subsets, the dataset is then split into a training set and a testing set, so that the split training set includes a plurality of subsets of the division, and the testing set includes a plurality of subsets of the division.

Process c2), for each methylation site, a target methylation site is determined based on the methylation data corresponding to the methylation sites in the plurality of subsets, respectively.

For the number of subsets into which the training set is divided, the embodiments of the present disclosure provide different methods for determining the target methylation site, as shown below.

Method c1, the training set includes two subsets, and the target methylation site is determined in the following manner.

For each methylation site, the target methylation site is determined based on the degree of difference in methylation data corresponding to the methylation site in each of the two subsets.

In embodiments, the degree of difference in the methylation data corresponding to the methylation site in each of the two subsets respectively is determined in the following manner:
performing a rank sum test on the methylation data corresponding to the methylation site in each of the two subsets, and determining the degree of difference based on a rank sum check value.

In the implementations, according to the cancer staging, the training set includes a subset 1 corresponding to the cancer staging as pre-stage and a subset 2 corresponding to the cancer staging as advanced stage, and the rank sum test is performed on the methylation data corresponding to the same methylation site in the two subsets to obtain the rank sum check value p. The formula is shown below:

$${P}_{n} = w ( {G}_{1} , {G}_{2} ) . \qquad Formula 1$$

In Formula 1, Pₙ is a rank sum test value (significance test value) obtained after performing the rank sum test on the n^{th} methylation site, W is the rank sum test function, G₁ denotes the methylation data of the n^{th} methylation site in subset 1, and G₂ denotes the methylation data of the n^{th} methylation site in subset 2.

Then, the top 1000 methylation sites with the highest degree of difference (i.e., those with the smallest p-value) are selected as the target methylation sites in order of p-value from smallest to largest. Theoretically, the methylation data corresponding to these 1000 methylation sites can more accurately distinguish target subjects under different cancer stages.

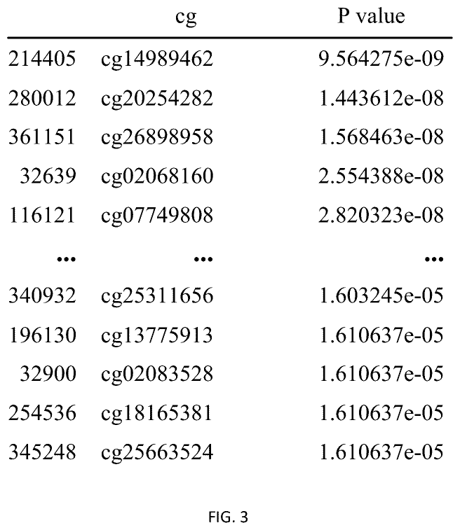

For example, the preprocessed dataset is classified according to early cancer (labeled 0), and advanced cancer (labeled 1), and two subsets are obtained, one subset corresponds to early cancer, and the other subset corresponds to advanced cancer, and the methylation data on each methylation site is differentially analyzed using the rank sum test, and the p-value is calculated. The p-value is sorted from smallest to largest, and the first 1000 methylation sites are selected as the target methylation sites. As shown in FIG. 3, a schematic diagram of a p-value for differential analysis of the methylation data corresponding to each methylation site provided in the embodiments of the present disclosure is shown. Here, the leftmost side indicates an index value of the methylation site, the column corresponding to cg indicates an identifier of the methylation site, and p-value indicates a rank sum test p-value obtained by calculating methylation data corresponding to the methylation site.

During analyzing the methylation data, the use of the rank sum test can detect differentially methylation sites, and since the number of sample sets under different cancer staging is usually different and the distribution of the methylation rate has no obvious regular pattern, the rank sum test can be used to perform a test of significance under the conditions that the data does not conform to a normal distribution and the number of sample sets is different, and the resulting methylation data corresponding to the most significant top 1000 target methylation sites is obtained.

Method c2, the training set includes more than two subsets, and the target methylation sites are determined in the following manner.

For each methylation site, the target methylation site is determined based on a correlation between the methylation data corresponding to the methylation site in each subset and the cancer staging value corresponding to the each subset.

In embodiments, the correlation between the methylation data corresponding to the methylation site in each subset and the cancer staging value corresponding to the subset is determined by:
performing a chi-square test on the methylation data corresponding to the methylation site in each subset and the cancer staging value corresponding to the subset, and determining the correlation based on a value of the chi-square test.

Here, the chi-square test is the degree of deviation between the actual observed values and the theoretically inferred values of the statistical samples. The degree of deviation between the actual observed values and the theoretically inferred values determines the magnitude of the chi-square value.

In the implementation, if the training set includes three or more subsets, each subset corresponds to a cancer stage, and the chi-square test can be used to calculate the correlation, i.e., the chi-square statistic, between each methylation site and the cancer stage value, and the 1000 methylation sites with the highest chi-square statistic are selected as the target methylation sites. The formula for calculating the chi-square test is shown below:

$${S}_{n} = chi 2 ( {X}_{n} , y ) . \qquad Formula 2$$

In Formula 2, Sn denotes the chi-square test value obtained after the chi-square test for the n^{th} methylation site, chi2 denotes the chi-square test function, Xₙ denotes the methylation data corresponding to the n^{th} methylation site in each subset, and y denotes the cancer staging value corresponding to each subset.

Here, when the training set is divided into three subsets, the value of y is 0, 1, 2; when the training set is divided into four subsets, the value of y is 0, 1, 2, 3.

Optionally, after the target methylation sites are determined as described above, the training set can also be preprocessed for standardization and dimensionality reduction, e.g., after 1000 target methylation sites are determined, the methylation data corresponding to the 1000 methylation sites is subjected to data standardization processing. The normalization process includes de-mean and variance normalization, the formula is as follows:

$${M}_{n}^{*} = \frac{{M}_{n} - μ}{σ} . \qquad Formula 3$$

In Formula 3, M*ₙ denotes the methylation data corresponding to the target methylation sites after the normalization process, Mₙ denotes the methylation data corresponding to the target methylation sites, μ denotes the mean value of each methylation data corresponding to the target methylation sites in each subset, and σ denotes the standard deviation of the methylation data corresponding to the target methylation sites in each subset.

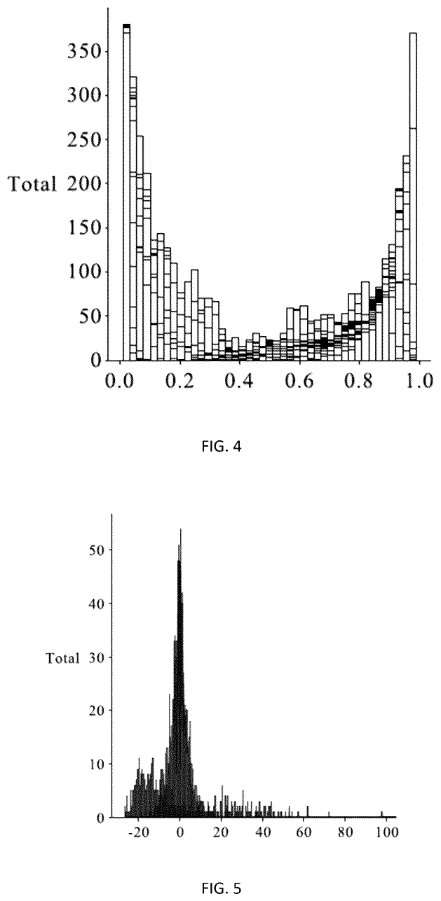

For example, the methylation data corresponding to 1000 target methylation sites are counted, and a distribution graph obtained is as shown in FIG. 4, which is a distribution graph of methylation data before normalization provided by the embodiments of the present disclosure, in which the distribution of methylation data corresponding to each target methylation site belongs to a skewed distribution, and the skewed distribution is not conducive to the training of the model, so the skewed data are normalized using the methods of de-mean and variance normalization, and the skewed data are obtained as close as possible to the normalized processing. Therefore, the skewed data is normalized using the methods of de-mean and variance normalization to obtain methylation data that converges to a normal distribution as much as possible for training the classification model.

Optionally, after the methylation data corresponding to the 1000 methylation sites is subjected to data normalization, the methylation data corresponding to the 1000 methylation sites can also be downsized to 10 feature data using the method of PCA downsizing. The formula is as follows:

$${X}^{*} = PCA ( n ) . fit\_transform ( X ) . \qquad Formula 4$$

In Formula 4, X* denotes the methylation data after the X dimensionality reduction process, X* can be understood as a matrix, PCA (n) denotes the PCA function that constructs the result of dimensionality reduction as n, n denotes the dimensionality of the methylation data after the dimensionality reduction, e.g., n=10, and the function fits and transfers X, and X denotes the methylation data before the dimensionality reduction.

In the implementations, since too many features will affect the model to find the intrinsic regular patterns, and the features of the methylation data in the training set in the implementations are unusually complex, the PCA dimensionality reduction method can be utilized to set the dimensionality to 10, and the high dimensional 1000 feature vectors (methylation data corresponding to 1000 methylation sites) can be dimensionalized down to low dimensional 10 feature vectors (methylation data corresponding to 10 methylation sites), which speeds up the training of the classification model and also ensures the characteristics of the metadata, as shown in FIG. 5, the embodiments of the present disclosure provide a distribution graph of the methylation data after standardization and dimensionality reduction processing, and the distribution of the methylation data after processing tends to be close to a normal distribution, which is more conducive to the training of the classification model.

Optionally, in order to adapt to the specific dataset analysis needs, the order of the rank sum test/chi-square test, the standardization treatment and the PCA dimensionality reduction can be freely switched. If a larger dataset, such as whole genome methylation sequencing data, is processed, since the number of methylation sites in the genome is generally in the thousands, PCA dimensionality reduction can be prioritized to reduce the dimensionality of the data and reduce the time for the rank sum test/chi-square test and standardization processing, and when the number of methylation sites is greater, the time saved is more obvious. In addition, if dealing with targeted methylation sequencing data containing only dozens of genes, then standardization can be performed first, followed by rank sum test/chi-square test and PCA dimensionality reduction, after standardization, the data distribution is more uniform, and when performing the test of significance, since it is not affected by the numerical size of the data itself, it can be easier to detect the differentially methylation sites, the sensitivity of rank sum test/chi-square test can be improved, and similarly the effect of PCA dimensionality reduction can be improved.

After processing the training set through the above process, the processed training set is input into the traversal fitting of the classification model algorithm, the training set is traversed into m classification models, and the model performance of each classification model is calculated. Optionally, the m classification models in the embodiments of the present disclosure include, but are not limited to, LogisticRegression, MLPClassifier, KNeighborsClassifier, Support Vector Machines (SVC), GaussianProcess (GaussianProcessClassifier), DecisionTreeClassifier, GaussianNB, RandomForestClassifier, DiscriminantAnalysis, MLPClassifier and other models. The classification accuracy of each classification model is calculated as an index for judging the performance of the model.

Step 2: according to the classification accuracies, k classification models are filtered out from the m classification models.

In the implementation, the first k classification models from the m classification models are selected for subsequent cross-validation and/or grid search (adjusting model parameters) in order of classification accuracy from largest to smallest.

Optionally, after selecting the k classification models from the m classification models according to the classification accuracies, any one or more of the following can also be performed:
a) filtering the n classification models from the k classification models using a cross-validation method, where m≥k≥n;
b) using the grid search method, adjusting the model parameters of the filtered classification models.

The above cross-validation and grid search can be executed simultaneously, but the order of execution can be customized according to actual needs. When the dataset is large, cross-validation can be performed first to filter out n classification models before using the grid search method to adjust the model parameters of the filtered out n classification models. When the dataset is small, the model parameters of the k classification models filtered out can be adjusted first using the grid search method, and then the n classification models can be filtered out from the k classification models using the cross-validation method.

In the implementations, the cross-validation method is utilized to filter out the n classification models from the k classification models, and the cross-validation formula is as follows:

$${C}_{k} = T ( {X}_{train} , {y}_{train} ) ; \qquad Formula 5 a$$

$${Score}_{k} = {C}_{k} ( {X}_{test} , {y}_{test} ) . \qquad Formula 5 b$$

Here, Xₜᵣₐᵢₙ, yₜᵣₐᵢₙ denote the methylation data in the training set and the corresponding cancer staging values in the training set, respectively; Xₜₑₛₜ, yₜₑₛₜ denote the methylation data in the testing set and the corresponding cancer staging values in the testing set, respectively; the training set and the testing set are the dataset segmentation under the k^{th} fold of the dataset, T denotes a variety of classification models referred to in the embodiments of the present disclosure, Cₖ denotes the classification model generated by fitting under the k^{th} fold, and Scoreₖ is the model AUC (evaluation index value) calculated by the classification model Cₖ under the k^{th} fold based on Xₜₑₛₜ, yₜₑₛₜ.

Optionally, cross-validation and filtering are performed in the following manner.

Step 3a), using the dataset, cross-validating each of the k classification models to obtain the respective evaluation metric values of the k classification models.

In the implementations, the model accuracy of the classification model can be used as the evaluation index value.

Step 3b), filtering n classification models from the k classification models based on the respective evaluation index values of the k classification models.

In the implementations, the first n classification models can be selected by arranging the classification models from high to low using the average AUC value of Scoreₖ as described above.

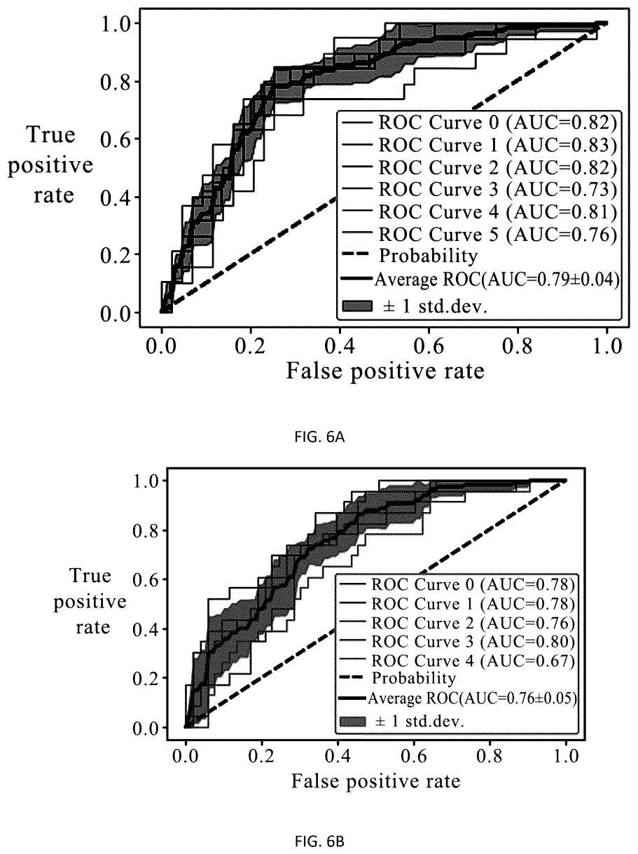

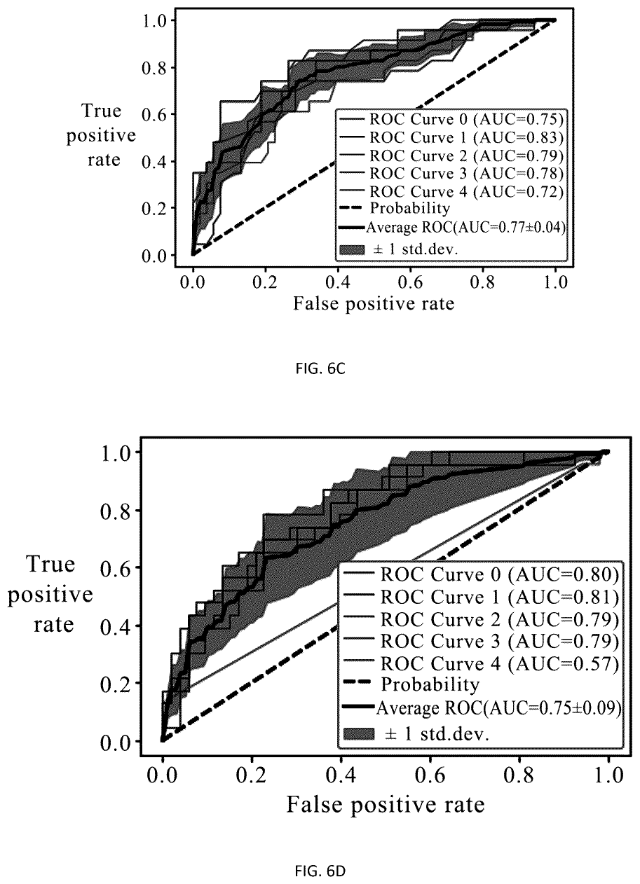

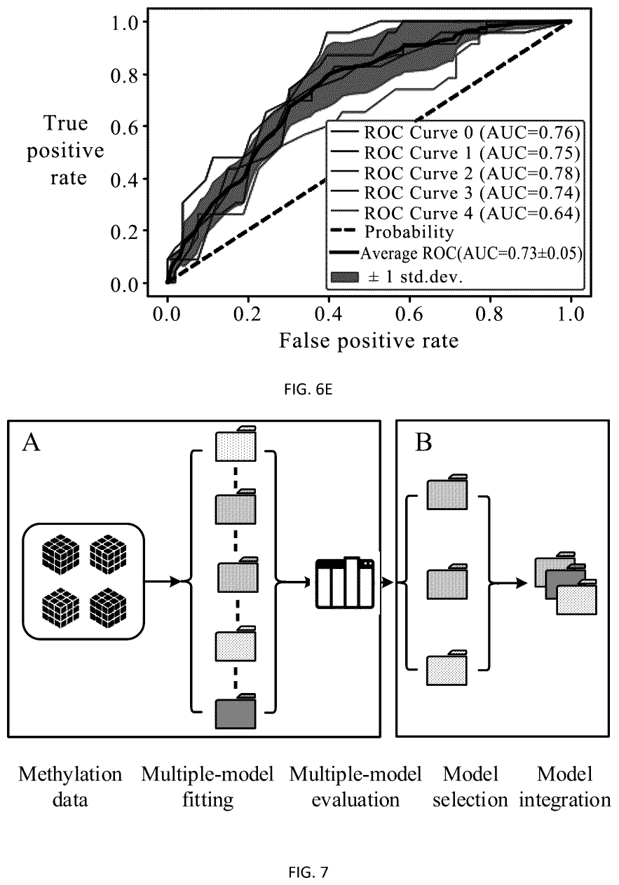

For example, to prevent over-fitting, a 5-fold cross-validation with K=5 is performed on the dataset, and the validation assessment method is selected as ROC_AUC and plotted, and the first 5 classification models with better cross validation are selected, including logistic regression, linear discriminant analysis, extreme random tree, Gaussian process, and the classification model with the closest neighbor of K has a better fitting method. As shown in FIGS. 6A to 6E, the embodiments of the present disclosure provide an evaluation schematic of cross-validation on the classification model, and FIGS. 6A to 6E show ROC_AUC diagrams of logistic regression, linear discriminant analysis, extreme random tree, Gaussian process, and K-nearest neighbor classification model, respectively.

The following parameter optimization process is also performed.

In some embodiments, the model parameters in the classification models obtained by filtering are adjusted using the grid search method to obtain the adjusted classification models. For example, the cross-validation is first performed, and then the grid search is performed, after obtaining the n classification models from k classification models by filtering using the cross-validation method, the model parameters of the classification models in the n classification models are is adjusted using the grid search method, and the final staging prediction model is determined according to the adjusted classification model.

In the implementations, after selecting the n classification models with better cross-validation, the model parameters of the classification models among the n classification models are tuned by grid search. The model parameters involved in the n classification models are arranged in combinations and configured, and the model parameter configurations of the optimal combinations are obtained based on the calculation results of the model accuracy.

For example, the classification models of logistic regression, linear discriminant analysis, extreme random tree, and Gaussian process mentioned above are tuned using the grid search method and evaluated using the ROC_AUC index. The tuned 4 classification models are subjected to subsequent model integration by logistic regression, linear discriminant analysis, Gaussian process, and extreme random tree.

In some embodiments, the staging prediction model is obtained by integrating according to the following:
the outputs of the n−1 classification models are used as an input of the n^{th} classification model, and the n classification models are integrated into the staging prediction model;
where the inputs of the n−1 classification models are the training set and the output of the n^{th} classification model is a cancer staging value corresponding to the training set.

In the implementations, the tuned n classification models are model integrated to generate a final staging prediction model. For example, when n=4, the features output from 3 classification models are fitted as input data to the fourth classification model to obtain the final staging prediction model. As shown in FIG. 7, the embodiments of the present disclosure provide a flowchart of a multi-model integration, in which methylation data in a training set is input to a plurality of classification models for fitting and training, the plurality of classification models are evaluated according to a model evaluation index, and n classification models are selected for model integration. The models in the figure are the classification models.

The formula for model integration is as follows:

$${C}_{combine} = {C}_{4} ( {S}_{C 1} , {S}_{C 2} , {S}_{C 3} ) . \qquad Formula 6$$

In Formula 6, C_{combine} denotes the integrated staging prediction model, S_{C1}, S_{C2}, S_{C3} denote prediction probability scores generated by the classification model C1, classification model C2, and classification model C3, respectively, and the final integrated model is obtained by inputting the output data of S_{C1}, S_{C2}, S_{C3} into the fourth classification model, C4, to perform model construction.

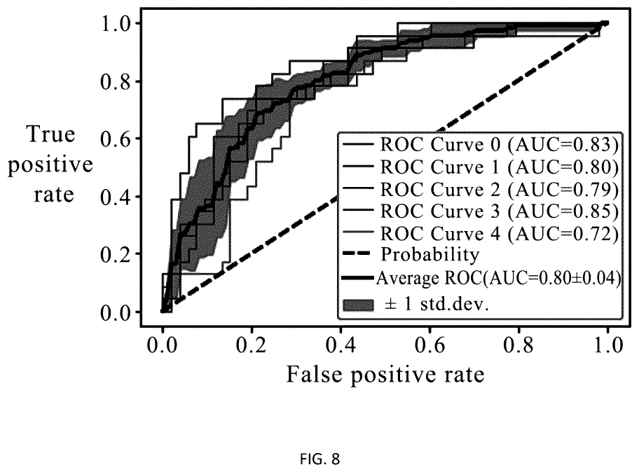

For example, four classification models (logistic regression, linear discriminant analysis, Gaussian process, and extreme random tree) that are tuned are selected for model integration using the stacking method in mlxtend, and the output data of the first three classification models are used as the input data of the last classification model to be fitted, and at the same time evaluated by using the ROC_AUC index, to generate a staging prediction model, the evaluation curve of the ROC_AUC index for the staging prediction model is shown in FIG. 8.

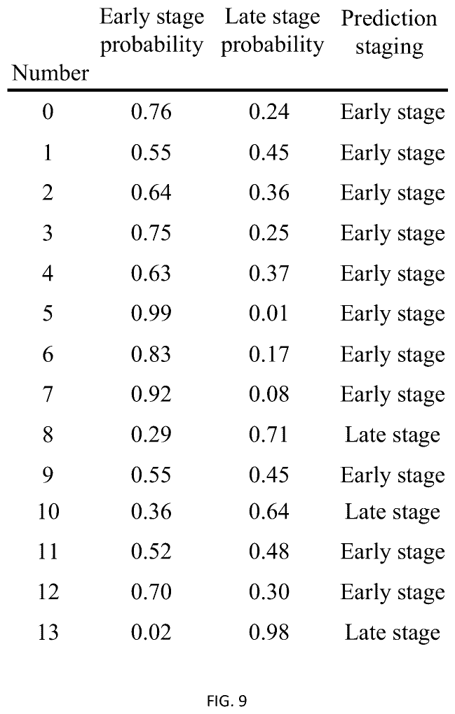

After the staging prediction model is integrated using the above approach, in the use phase, the methylation data of patients with unknown liver cancer stages are obtained, then 1000 target methylation sites corresponding to the staging prediction model are selected, and finally the methylation data corresponding to the 1000 target methylation sites are normalized and dimensionally downgraded, and the normalized and dimensionally downgraded methylation data are inputted to the staging prediction model for staging prediction, and the corresponding prediction results are output, as shown in FIG. 9, the embodiments of the present disclosure provide a graph of the results of cancer staging prediction of liver cancer patients using the staging prediction model, which contains the predicted staging of each liver cancer patient, and the probability of the specific staging is indicated.

In the embodiments of the present disclosure, traversal fitting classification algorithms (using a training set to train m classification models independently), cross-validation, and grid search tuning are used cooperatively. Traversing the fitting can initially select candidate superior classification algorithms among all the classification algorithms, and cross-validation can assess whether the aforementioned candidate classification algorithms are performing well across the entire dataset, and is used to assess the stability of the classification model and its generalization, for example, if the candidate classification model selected by traversal fitting fluctuates greatly in the AUC index of all folds of cross-validation, then the classification algorithm used for that classification model will be excluded, model tuning is to select the parameters that obtain the best performance of the model based on the n classification models that have already been selected, after traversal fitting classification algorithms, cross-validation and grid search tuning of the classification model, its accuracy, stability and predictive ability for new datasets are optimal. Model integration is used to further integrate the aforementioned better n models to obtain a final staging prediction model, and the final staging prediction model has a higher performance than any one of the n models.

Optionally, if the amount of data is not large, such as dealing with targeted methylation sequencing data containing only dozens of genes, the modeling can be modeled along the lines of traversal fitting classification model, grid search, and then cross-validation, and such operations can evaluate all the classification algorithms and their belonging parameters, and theoretically consider the most adequate conditions and the most comprehensive consideration in filtering the model. However, because the computational complexity of considering all classification algorithms and their affiliated parameters increases exponentially, it consumes the longest time. If the amount of data is large, such as when dealing with whole genome methylation sequencing data, it can be modeled by the concept of traversal fitting classification model, then cross-validation, and finally grid search, traversal fitting classification model can initially select the better classification algorithm among all the classification algorithms, cross-validation is used to assess the stability of the classification model and its ability to generalize, and grid search (i.e., model tuning) is designed to select the parameters to obtain the best performance of the model based on the selected n classification models, and the classification model after traversal fitting classification algorithm, cross-validation, and grid search tuning has the best accuracy, stability, and prediction ability for new datasets.

The concept of model integration in the embodiments of the present disclosure is to fit the output data of the first three classification models as the input data of the last classification model to obtain the final staging prediction model. Specifically, the methylation data after normalization and dimensionality reduction processing is input into each of the three classification models, and the prediction results of the prediction probability of each class output from the three classification models are used as an original feature data, and the original feature data is again input into the fourth classification model for modeling. The method of modeling using the features generated by the first three classifiers as the input data of the last classifier, due to the use of the specific values of the probability of each classifier under different classifications and the use of the probability values as the original dataset for the evaluation of the model construction, compared with the method of voting-weighted integration, the method is a more complete synthesis of the different classification models, and it can be objectively considered the weight of each classifier in the prediction results, which theoretically improves the accuracy of the final model. The voting-weighted integration method only makes the final judgment from the statistics of the percentage of classification results of different classifiers, which exacerbates the influence of the less effective classifiers on the final results and introduces part of the noise when the weights of the different classifiers are not taken into account, and the setting of the size of the weights of the different classifiers is more subjective when the weights of the different classifiers are taken into account.

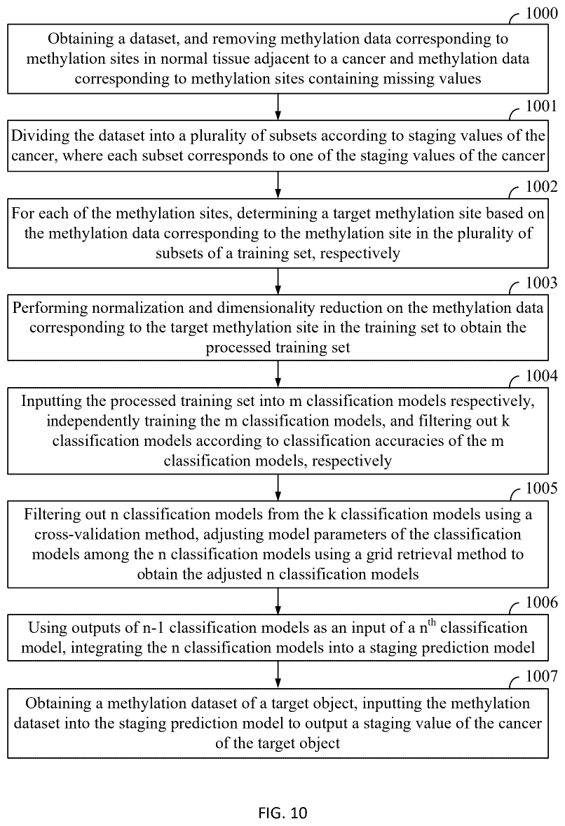

As shown in FIG. 10, a specific implementation process of a cancer staging method provided by the embodiments of the present disclosure is shown below.

Step 1000, obtaining a dataset, and removing methylation data corresponding to methylation sites in normal tissue adjacent to a cancer and methylation data corresponding to methylation sites containing missing values.

Step 1001, dividing the dataset into a plurality of subsets according to cancer staging values, where each subset corresponds to one of the cancer staging values.

Step 1002, for each of the methylation sites, determining a target methylation site based on the methylation data corresponding to the methylation site in the plurality of subsets of a training set, respectively.

Step 1003, performing normalization and dimensionality reduction on the methylation data corresponding to the target methylation site in the training set to obtain the processed training set.

Step 1004: inputting the processed training set into m classification models respectively, independently training the m classification models, and filtering out k classification models according to classification accuracies of the m classification models, respectively.

Step 1005: filtering out n classification models from the k classification models using a cross-validation method, adjusting model parameters of the classification models among the n classification models using a grid search method to obtain the adjusted n classification models.

Step 1006, using outputs of n−1 classification models as an input of a n^{th} classification model, integrating the n classification models into a staging prediction model.

Step 1007, obtaining a methylation dataset of a target object, inputting the methylation dataset into the staging prediction model to output a staging value of the cancer of the target object.

In the cancer staging method provided in the embodiments of the present disclosure, the methylation data of the cancer patient and the corresponding cancer staging value are obtained to form a dataset, the dataset is pre-processed and divided into subsets; subsequently, the dataset is split into a training set and a testing set in a ratio of 8:2, a differential methylation analysis is performed on the training set, a plurality of target methylation sites with the highest degree of difference are selected, and a PCA method is used to perform dimensionality reduction on methylation data corresponding to the plurality of target methylation sites using PCA method; traversal fitting the training set using a variety of classification algorithms, the fitted classification model is used for testing in the testing set, the n classification models with better test results are selected for grid search tuning, and integration is performed according to the tuned classification models to obtain a final staging prediction model; and the final staging prediction model is used for staging prediction of the cancer of the patient. The embodiments of the present disclosure utilizes methylation site information for cancer staging prediction, which is applicable to the vast majority of cancer types compared to current clinical staging methods, and can effectively differentiate cancer staging types of different patients. Combined with the classification algorithm of differential methylation site filtering, feature dimensionality reduction and multi-model integration, it can effectively capture the complex relationship between methylation data and cancer staging and improve the accuracy of cancer staging prediction.

Based on the same inventive concept, the embodiments of the present disclosure also provide a cancer staging device, and since the device is the device in the method in the embodiments of the present disclosure, and the device solves the problem in a similar way as the method, the implementation of the device can be seen in the implementation of the method, and the repetition will not be repeated.

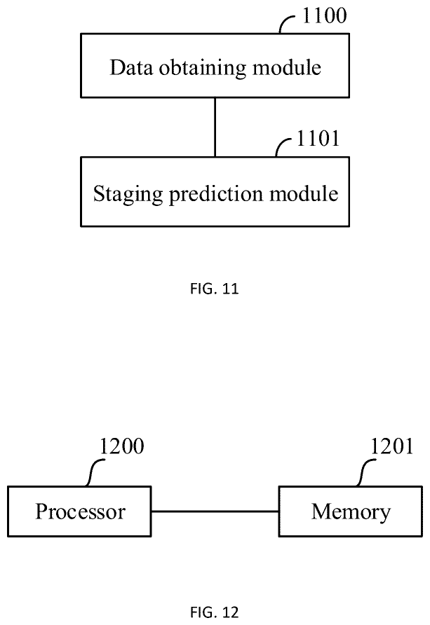

As shown in FIG. 11, the device includes:
a data obtaining module 1100 for obtaining a methylation dataset of a target object;
a staging prediction module 1101 for inputting the methylation dataset into a staging prediction model to output a cancer staging value of the target object; where the staging prediction model is obtained by integrating n classification models, model parameters of the n classification models are obtained by optimization using a grid search method, the n classification models are obtained by filtering from m classification models using a cross-validation method, the m classification models are obtained by independent training using a same dataset, wherein m>n, m is an integer greater than 2, and n is an integer greater than 1.

As an optional embodiment, the data obtaining module 1100 is specifically used for:
obtaining methylation data corresponding to a target methylation site of the target object, and determining the methylation dataset based on the methylation data corresponding to the target methylation site;
wherein the target methylation site is determined based on a degree of difference in methylation data of a same methylation site corresponding to different cancer staging values, or a correlation between methylation data corresponding to a methylation site and different cancer staging values.

As an optional embodiment, the staging prediction model further includes a normalization model, and the normalization model is used to normalize methylation data in the methylation dataset by means of de-meaning and variance normalization.

As an optional embodiment, the staging prediction model further includes a dimensionality reduction model, and the dimensionality reduction model is used to perform dimensionality reduction on methylation data in the methylation dataset according to a principal component analysis (PCA) method.

As an optional embodiment, the staging prediction module 1101 is specifically used to determine the staging prediction model by:
independently training the m classification models separately using the dataset, and determining classification accuracies of the m classification models;
filtering out k classification models from the m classification models according to the classification accuracies.

As an optional embodiment, after filtering out the k classification models from the m classification models according to the classification accuracies, the staging prediction module 1101 is specifically further used for:
filtering out the n classification models from the k classification models using the cross-validation method, wherein m≥k≥n; and/or
adjusting the model parameters of the filtered classification models using the grid search method.

As an optional embodiment, the staging prediction module 1101 is specifically used for:
cross-validating the k classification models using the dataset to obtain evaluation index values of the k classification models;
filtering out the n classification models from the k classification models based on the evaluation index values of the k classification models.

As an optional embodiment, after obtaining the dataset, the data obtaining module 1100 is specifically further used for:
filtering out methylation data corresponding to a target methylation site from methylation data contained in the dataset, and independently training the m classification models using the filtered methylation data.

As an optional embodiment, the dataset includes sample sets of different target objects, the sample sets each includes methylation data corresponding to each methylation site, and one of the sample sets corresponds to one of the cancer staging values; the data obtaining module 1100 is specifically used for determining the target methylation site by:
dividing the dataset into a plurality of subsets according to the cancer staging values corresponding to the sample sets, wherein one of the subsets corresponds to one of the cancer staging values, and sample sets contained in different subsets correspond to different cancer staging values;
for each methylation site, determining the target methylation site according to the methylation data in the plurality of subsets corresponding to the methylation site, respectively.

As an optional embodiment, the data obtaining module 1100 is specifically used for:
when the dataset is divided into two subsets, determining, for each methylation site, the target methylation site according to a degree of difference in the methylation data corresponding to the methylation site in each of the two subsets; or
when the dataset is divided into more than two subsets, determining, for each methylation site, the target methylation site according to a correlation between the methylation data corresponding to the methylation site in each subset and the cancer staging value corresponding to the each subset.

As an optional embodiment, the data obtaining module 1100 is specifically used to determine the degree of difference in the methylation data corresponding to the methylation site in each of the two subsets by:
performing a rank sum test on the methylation data corresponding to the methylation site in each of the two subsets, and determining the degree of difference according to a rank sum check value; or
the correlation between the methylation data corresponding to the methylation site in each subset and the cancer staging value corresponding to the each subset is determined by:
performing a chi-square test on the methylation data corresponding to the methylation site in each subset and the cancer staging value corresponding to the subset, and determining the correlation according to a value of the chi-square test.

As an optional embodiment, the plurality of subsets includes more than two subsets; the data obtaining module 1100 is specifically further used for:
when a subset in which a quantity of sample sets is lower than a threshold value exists in the divided plurality of subsets, merging the subset into another subset according to the cancer staging value corresponding to the subset, determining a cancer staging value corresponding to the another subset after the merging, and determining the target methylation site and training of the classification model using the subset after the merging; wherein the cancer staging value corresponding to the another subset is closest to the cancer staging value corresponding to the subset.

As an optional embodiment, the data obtaining module 1100 is specifically further used for:
removing, with respect to a sample set of the target object, methylation data corresponding to a methylation site of normal tissue adjacent to a cancer in the sample set, and/or, methylation data corresponding to a methylation site containing a missing value.

As an optional embodiment, the staging prediction model is obtained by integrating according to the following:
integrating the n classification models into the staging prediction model by using outputs of n−1 classification models as an input of an n^{th} classification model;
wherein inputs of the n−1 classification models are the dataset, and an output of the n^{th} classification model is a cancer staging value corresponding to the dataset.

Based on the same inventive concept, the embodiments of the present disclosure also provide an electronic device, and since the electronic device is the electronic device in the method in the embodiments of the present disclosure, and the principle of solving problem of the electronic device is similar to that of the method, the implementation of the electronic device can be seen in the implementation of the method, and the repetition will not be repeated.

As shown in FIG. 12, the electronic device includes a processor 1200 and a memory 1201, the memory 1201 is used to store programs executable by the processor 1200, and the processor 1200 is used to read the programs in the memory 1201 and perform the following steps:
obtaining a methylation dataset of a target object;
inputting the methylation dataset into a staging prediction model to output a cancer staging value of the target object;
wherein the staging prediction model is obtained by integrating n classification models, model parameters of the n classification models are obtained by optimization using a grid search method, the n classification models are obtained by filtering from m classification models using a cross-validation method, the m classification models are obtained by independent training using a same dataset, wherein m>n, m is an integer greater than 2, and n is an integer greater than 1.

As an optional embodiment, the processor 1200 is specifically configured to perform:
obtaining methylation data corresponding to a target methylation site of the target object, and determining the methylation dataset based on the methylation data corresponding to the target methylation site;
wherein the target methylation site is determined based on a degree of difference in methylation data of a same methylation site corresponding to different cancer staging values, or a correlation between methylation data corresponding to a methylation site and different cancer staging values.

As an optional embodiment, the staging prediction model further includes a normalization model, the normalization model is used to normalize the methylation data in the methylation dataset by means of de-meaning and variance normalization.

As an optional embodiment, the staging prediction model further includes a dimensionality reduction model, and the dimensionality reduction model is used to perform dimensionality reduction on methylation data in the methylation dataset according to a principal component analysis (PCA) method.

As an optional embodiment, the processor 1200 is specifically configured to determine the staging prediction model by:
independently training the m classification models separately using the dataset, and determining classification accuracies of the m classification models;
filtering out k classification models from the m classification models according to the classification accuracies.

As an optional embodiment, after filtering out the k classification models from the m classification models according to the classification accuracies, the processor 1200 is specifically further configured to perform:
filtering out the n classification models from the k classification models using the cross-validation method, wherein m≥k≥n; and/or
adjusting the model parameters of the filtered classification models using the grid search method.

As an optional embodiment, the processor 1200 is specifically configured to perform: cross-validating the k classification models using the dataset to obtain evaluation index values of the k classification models;
filtering out the n classification models from the k classification models based on the evaluation index values of the k classification models.

As an optional embodiment, after obtaining the dataset, the processor 1200 is specifically further configured to perform:
filtering out methylation data corresponding to a target methylation site from methylation data contained in the dataset, and independently training the m classification models using the filtered methylation data.

As an optional embodiment, the dataset includes sample sets of different target objects, the sample sets each includes methylation data corresponding to each methylation site, and one of the sample sets corresponds to one of the cancer staging values; the processor 1200 is specifically configured to determine the target methylation site by:
dividing the dataset into a plurality of subsets according to the cancer staging values corresponding to the sample sets, wherein one of the subsets corresponds to one of the cancer staging values, and sample sets contained in different subsets correspond to different cancer staging values;
for each methylation site, determining the target methylation site according to the methylation data in the plurality of subsets corresponding to the methylation site, respectively.

As an optional embodiment, the processor 1200 is specifically configured to perform:
when the dataset is divided into two subsets, determining, for each methylation site, the target methylation site according to a degree of difference in the methylation data corresponding to the methylation site in each of the two subsets; or
when the dataset is divided into more than two subsets, determining, for each methylation site, the target methylation site according to a correlation between the methylation data corresponding to the methylation site in each subset and the cancer staging value corresponding to the each subset.

As an optional embodiment, the processor 1200 is specifically configured to determine the degree of difference in the methylation data corresponding to the methylation site in each of the two subsets by:
performing a rank sum test on the methylation data corresponding to the methylation site in each of the two subsets, and determining the degree of difference according to a rank sum check value; or.
the correlation between the methylation data corresponding to the methylation site in each subset and the cancer staging value corresponding to the each subset is determined by:
performing a chi-square test on the methylation data corresponding to the methylation site in each subset and the cancer staging value corresponding to the subset, and determining the correlation according to a value of the chi-square test.

As an optional embodiment, the plurality of subsets include more than two subsets; the dividing the dataset into the plurality of subsets according to the cancer staging values corresponding to the sample sets, the processor 1200 is specifically further configured to perform:
when a subset in which a quantity of sample sets is lower than a threshold value exists in the divided plurality of subsets, merging the subset into another subset according to the cancer staging value corresponding to the subset, determining a cancer staging value corresponding to the another subset after the merging, and determining the target methylation site and training of the classification model using the subset after the merging; wherein the cancer staging value corresponding to the another subset is closest to the cancer staging value corresponding to the subset.

As an optional embodiment, the processor 1200 is specifically further configured to perform:
removing, with respect to a sample set of the target object, methylation data corresponding to a methylation site of normal tissue adjacent to a cancer in the sample set, and/or, methylation data corresponding to a methylation site containing a missing value.

As an optional embodiment, the staging prediction model is obtained by integrating according to the following:
integrating the n classification models into the staging prediction model by using outputs of n−1 classification models as an input of an n^{th} classification model;
wherein inputs of the n−1 classification models are the dataset, and an output of the n^{th} classification model is a cancer staging value corresponding to the dataset.

Based on the same inventive concept, embodiments of the present disclosure provide a computer storage medium, the computer storage medium including: computer program codes which, when the computer program codes are run on a computer, cause the computer to execute a cancer staging method as any of the foregoing discussed. Since the principle of problem solving of the above-described computer storage medium is similar to the cancer staging method, the implementation of the above-described computer storage medium can be seen in the implementation of the method, and the repetition will not be repeated.

In a specific implementation, the computer storage medium may include: a Universal Serial Bus Flash Drive (USB), a removable hard disk, a Read-Only Memory (ROM), a Random Access Memory (RAM), Disk or CD-ROM, and various other storage media that can store program codes.

Based on the same inventive concept, embodiments of the present disclosure also provide a computer program product including: a computer program code which, when run on a computer, causes the computer to perform the cancer staging method as any of the foregoing. Since the above-described computer program product solves the problem in a similar way to the cancer staging method, the implementation of the above-described computer program product can be seen in the implementation of the method, and the repetition will not be repeated.

The computer program product may employ any combination of one or more readable media. The readable medium can be a readable signal medium or a readable storage medium. The readable storage medium may, for example, be, but is not limited to, a system, apparatus, or device that is electrical, magnetic, optical, electromagnetic, infrared, or semiconducting, or any combination of the above. More specific examples of readable storage media (a non-exhaustive list) include: an electrical connection having one or more wires, a portable disk, a hard disk, a random access memory (RAM), a read-only memory (ROM), an erasable programmable read-only memory (EPROM or flash memory), a fiber optic, a portable compact disk read-only memory (CD-ROM), an optical storage device, a magnetic storage device, or any suitable combination of the foregoing.

It should be appreciated by those skilled in the art that embodiments of the present disclosure can be provided as methods, systems, or computer program products. Thus, the present disclosure may take the form of a fully hardware embodiment, a fully software embodiment, or an embodiment that combines software and hardware aspects. Further, the present disclosure may take the form of a computer program product implemented on one or more computer-usable storage media (including, but not limited to, disk memory and optical memory, etc.) that contain computer-usable program code therein.

The present disclosure is described with reference to flowcharts and/or block diagrams of methods, devices (systems), and computer program products according to embodiments of the present disclosure. It is to be understood that each of the processes and/or boxes in the flowcharts and/or block diagrams, and combinations of the processes and/or boxes in the flowcharts and/or block diagrams, can be implemented by computer program instructions. These computer program instructions can be provided to a processor of a general-purpose computer, a special-purpose computer, an embedded processor, or other programmable data-processing device to produce a machine such that the instructions executed by the processor of the computer or other programmable data-processing device produce a device for carrying out the functions specified in the one process or multiple processes of the flowchart and/or the one box or multiple boxes of the box diagram.

These computer program instructions can also be stored in computer-readable memory capable of directing the computer or other programmable data processing device to operate in a particular manner such that the instructions stored in that computer-readable memory produce an article of manufacture including an instructional device that implements the function specified in the flowchart one process or a plurality of processes and/or the box diagram one box or a plurality of boxes.

These computer program instructions can also be loaded onto a computer or other programmable data processing device such that a series of operational steps are performed on the computer or other programmable device to produce computer-implemented processing, such that the instructions executed on the computer or other programmable device provide steps for implementing the functionality specified in the flowchart one process or a plurality of processes and/or the box diagram one box or a plurality of boxes.

Obviously, those skilled in the art can make various changes and variations to the present disclosure without departing from the spirit and scope of the present disclosure. Thus, to the extent that such modifications and variations of the present disclosure are within the scope of the present claims and their technical equivalents, the present disclosure is intended to encompass such modifications and variations.

1. A cancer staging method, comprising:
obtaining a methylation dataset of a target object;
inputting the methylation dataset into a staging prediction model to output a cancer staging value of the target object;
wherein the staging prediction model is obtained by integrating n classification models, model parameters of the n classification models are obtained by optimization using a grid search method, the n classification models are obtained by filtering from m classification models using a cross-validation method, the m classification models are obtained by independent training using a same dataset, wherein m>n, m is an integer greater than 2, and n is an integer greater than 1.

2. The method according to claim 1, wherein the obtaining the methylation dataset of the target object comprises:
obtaining methylation data corresponding to a target methylation site of the target object, and determining the methylation dataset based on the methylation data corresponding to the target methylation site;
wherein the target methylation site is determined based on a degree of difference in methylation data of a same methylation site corresponding to different cancer staging values, or a correlation between methylation data corresponding to a methylation site and different cancer staging values.

3. The method according to claim 1, wherein the staging prediction model further comprises a normalization model, and the normalization model is used to normalize methylation data in the methylation dataset by means of de-meaning and variance normalization.

4. The method according to claim 1, wherein the staging prediction model further comprises a dimensionality reduction model, and the dimensionality reduction model is used to perform dimensionality reduction on methylation data in the methylation dataset according to a principal component analysis (PCA) method.

5. The method according to claim 1, wherein the staging prediction model is determined by:
independently training the m classification models separately using the dataset, and determining classification accuracies of the m classification models;
filtering out k classification models from the m classification models according to the classification accuracies.

6. The method according to claim 5, wherein after filtering out the k classification models from the m classification models according to the classification accuracies, the method further comprises:
filtering out the n classification models from the k classification models using the cross-validation method, wherein m≥k≥n; and/or
adjusting the model parameters of the filtered classification models using the grid search method.

7. The method according to claim 6, wherein the filtering out the n classification models from the k classification models using the cross-validation method comprises:
cross-validating the k classification models using the dataset to obtain evaluation index values of the k classification models;
filtering out the n classification models from the k classification models based on the evaluation index values of the k classification models.

8. The method according to claim 5, wherein after obtaining the dataset, the method further comprises:
filtering out methylation data corresponding to a target methylation site from methylation data contained in the dataset, and independently training the m classification models using the filtered methylation data.

9. The method according to claim 2, wherein the dataset comprises sample sets of different target objects, the sample sets each comprises methylation data corresponding to each methylation site, and one of the sample sets corresponds to one of the cancer staging values;
wherein the target methylation site is determined by:
dividing the dataset into a plurality of subsets according to the cancer staging values corresponding to the sample sets, wherein one of the subsets corresponds to one of the cancer staging values, and sample sets contained in different subsets correspond to different cancer staging values;
for each methylation site, determining the target methylation site according to the methylation data in the plurality of subsets corresponding to the methylation site, respectively.

10. The method according to claim 9, wherein the determining the target methylation site according to the methylation data in the plurality of subsets corresponding to the methylation site, respectively, comprises:
when the dataset is divided into two subsets, determining, for each methylation site, the target methylation site according to a degree of difference in the methylation data corresponding to the methylation site in each of the two subsets; or.
when the dataset is divided into more than two subsets, determining, for each methylation site, the target methylation site according to a correlation between the methylation data corresponding to the methylation site in each subset and the cancer staging value corresponding to the each subset.

11. The method according to claim 10,
wherein the degree of difference in the methylation data corresponding to the methylation site in each of the two subsets is determined by:
performing a rank sum test on the methylation data corresponding to the methylation site in each of the two subsets, and determining the degree of difference according to a rank sum check value; or
the correlation between the methylation data corresponding to the methylation site in each subset and the cancer staging value corresponding to the each subset is determined by:
performing a chi-square test on the methylation data corresponding to the methylation site in each subset and the cancer staging value corresponding to the subset, and determining the correlation according to a value of the chi-square test.

12. The method according to claim 9, wherein the plurality of subsets comprise more than two subsets; the dividing the dataset into the plurality of subsets according to the cancer staging values corresponding to the sample sets, further comprises:
when a subset in which a quantity of sample sets is lower than a threshold value exists in the divided plurality of subsets, merging the subset into another subset according to the cancer staging value corresponding to the subset, determining a cancer staging value corresponding to the another subset after the merging, and determining the target methylation site and training of the classification model using the subset after the merging; wherein the cancer staging value corresponding to the another subset is closest to the cancer staging value corresponding to the subset.

13. The method according to claim 9, further comprising:
removing, with respect to a sample set of the target object, methylation data corresponding to a methylation site of normal tissue adjacent to a cancer in the sample set, and/or, methylation data corresponding to a methylation site containing a missing value.

14. The method according to claim 1, wherein the staging prediction model is obtained by integrating according to the following:
integrating the n classification models into the staging prediction model by using outputs of n−1 classification models as an input of an n^{th} classification model;
wherein inputs of the n−1 classification models are the dataset, and an output of the n^{th} classification model is a cancer staging value corresponding to the dataset.

15. An electronic device, comprising a processor and a memory, wherein the memory is configured for storing programs executable by the processor, and the processor is configured for reading the programs in the memory to perform:
obtaining a methylation dataset of a target object;
inputting the methylation dataset into a staging prediction model to output a cancer staging value of the target object;
wherein the staging prediction model is obtained by integrating n classification models, model parameters of the n classification models are obtained by optimization using a grid search method, the n classification models are obtained by filtering from m classification models using a cross-validation method, the m classification models are obtained by independent training using a same dataset, wherein m>n, m is an integer greater than 2, and n is an integer greater than 1.

16. A computer storage medium, storing computer programs, wherein the programs when executed by a processor implement:
obtaining a methylation dataset of a target object;
inputting the methylation dataset into a staging prediction model to output a cancer staging value of the target object;
wherein the staging prediction model is obtained by integrating n classification models, model parameters of the n classification models are obtained by optimization using a grid search method, the n classification models are obtained by filtering from m classification models using a cross-validation method, the m classification models are obtained by independent training using a same dataset, wherein m>n, m is an integer greater than 2, and n is an integer greater than 1.

17. The electronic device according to claim 15, wherein the processor is further configured for reading the programs in the memory to perform:
obtaining methylation data corresponding to a target methylation site of the target object, and determining the methylation dataset based on the methylation data corresponding to the target methylation site;
wherein the target methylation site is determined based on a degree of difference in methylation data of a same methylation site corresponding to different cancer staging values, or a correlation between methylation data corresponding to a methylation site and different cancer staging values.

18. The electronic device according to claim 15, wherein the staging prediction model further comprises a normalization model, and the normalization model is used to normalize methylation data in the methylation dataset by means of de-meaning and variance normalization.

19. The electronic device according to claim 15, wherein the staging prediction model further comprises a dimensionality reduction model, and the dimensionality reduction model is used to perform dimensionality reduction on methylation data in the methylation dataset according to a principal component analysis (PCA) method.

20. The electronic device according to claim 15, wherein the processor is further configured for reading the programs in the memory to determine the staging prediction model by:
independently training the m classification models separately using the dataset, and determining classification accuracies of the m classification models;
filtering out k classification models from the m classification models according to the classification accuracies.
